(19) United States
(12) Patent Application Publication
(10) Pub. No.: US 2020/0306496 A1
(43) Pub. Date: Oct. 1, 2020

(54) METHOD AND SYSTEM FOR DELIVERING SENSORY SIMULATION TO A USER

(71) Applicant: KONINKLIJKE PHILIPS N.V., Eindhoven (NL)

(72) Inventors: Mustafa Ghassan RADHA, Veldhoven (NL); Pedro Miguel FERREIRA DOS SANTOS DA FONSECA, Borgerhout (BE); Stefan PFUNDTNER, Eindhoven (NL)

(21) Appl. No.: 16/832,927

(22) Filed: Mar. 27, 2020

Related U.S. Application Data
(60) Provisional application No. 62/826,103, filed on Mar. 29, 2019.

(51) Int. Cl. A61M 21/02

(52) U.S. Cl. CPC A61M 21/02; A61M 2021/0016

(57) ABSTRACT

A system for delivering sensory stimulation comprises a sensor configured to measure autonomic nervous system activity information of a user during a sleep session; a sensory stimulator configured to deliver sensory simulation to the user during the sleep session; and a computer system. One or more physical processors of the computer system are programmed with computer program instructions which, when executed cause the computer system to: obtain the autonomic nervous system activity information of the user; determine parasympathetic nervous system information based on the obtained autonomic nervous system activity information of the user; and provide input to the sensory stimulator based on the determined parasympathetic nervous system information, the provided input causing the sensory stimulator to deliver the sensory stimulation to the user based on the determined parasympathetic nervous system information.

### CROSS-REFERENCE TO PRIOR APPLICATIONS

This application claims the benefit of U.S. Provisional Application No. 62/826103, filed on 29 Mar. 2019. This application is hereby incorporated by reference herein.

### BACKGROUND

### 1. Field

The present patent application discloses a method and a system for delivering sensory stimulation to a user, specifically, a method and a system for providing sleep enhancement through slow wave stimulation based on autonomic nervous system activity.

### 2. Description of the Related Art

Sleep is a reversible state of disconnection from the external environment, characterized by quiescence and reduced vigilance. The state of the brain and body are not uniform throughout a complete night of sleep, a fact that many believe means that sleep has multiple purposes.

Systems for monitoring sleep are known. Known systems detect sleep stages in a subject during a sleep session. The American Academy of Sleep Medicine (AASM) identifies sleep to be composed of two distinct types: Rapid-Eye Movement (REM) Sleep and Non-Rapid Eye Movement (NREM) Sleep. The AASM further identifies three stages of NREM sleep, called NREM1, NREM2 and NREM3, with 1 being the lightest NREM sleep and 3 being the deepest NREM sleep. Deeper NREM sleep is associated with larger quantities of slow wave activity (SWA) in the EEG (electroencephalogram).

Slow wave activity has been explained through the Synaptic Homeostasis Hypothesis (SHH). The SHH hypothesis states that storing new information regarding to daily experiences while awake requires electricity to potentiate synapses. Slow waves are then the observable of the brain when the brain is de-potentiating synapses, consolidating experiences and removing unnecessary information. This process is referred to as process S, a distinct circadian process. That is, the exemplary circadian process includes homeostatic sleep drive (process S) and circadian drive for arousal (process C).

Over the last decade, it has been shown in multiple studies that the use of auditory stimulation during sleep can enhance sleep slow waves, both in size as well as quantity, which both contribute to synaptic depotentiation. Early results indicate that participants undergoing such stimulation feel they sleep better and have better cognitive capacity (e.g., they perform better on memory puzzles after the stimulation).

However, a critical problem is that the stimulation may not happen when subjects are not sleeping deeply enough. If subjects are still awake, studies show that people will take longer to fall asleep. Other experiments show that if the person is not sleeping deep enough, the audio simulation will wake them up (by causing a cortical arousal). This is why in the current solution the auditory stimulation is limited to NREM3, the deepest NREM sleep stage, thereby preventing stimulation when the user is vigilant enough to hear the sound. This is the core functionality of a product that detects NREM3 through brain activity and starts stimulation through built-in headphones (see FIG. 1). To detect NREM3, an EEG sensor is included in the cap that is processed by an algorithm.

One problem with the product/system shown and described with respect to FIG. 1 is that the use of a head cap can be obtrusive and, therefore, impair sleep quality, which is counterproductive to the purpose of the product (i.e., enhancing the sleep quality). While the use of other signals (e.g., cardiorespiratory signals) instead of EEG signals may alleviate the ergonomic issues of the product/system shown and described with respect to FIG. 1, there remains a second unaddressed problem: the use of NREM3 as the stimulation period is not always optimal. This is because of these reasons: 1) slow waves often also be evoked in other sleep stages (i.e., this is a missed out stimulation opportunity); 2) arousability may not always be the lowest during NREM3 ; and 3) NREM3 is a sleep stage that becomes scarcer as humans age, making the product/system shown and described with respect to FIG. 1 less effective for older users. However, these people could benefit from stimulation in other sleep stages when their arousability is low. This is caused mainly by the fact that the hallmark of NREM3, slow waves in the EEG signals, become lower in amplitude with advancing age, making them more difficult to visually recognize, automatically detect, or simply not surpass the slow wave amplitude criteria specified by the AASM.

The reason for this is that the definition of NREM3 is based on slow wave activity itself. It is, in fact, defined as a thirty second period of sleep in which the number of slow waves in the EEG signals exceed a certain threshold. However, slow waves can also already occur in NREM2 (e.g., at a lower number that does not meet the threshold). While arousability is typically low during NREM3, there are other markers for arousal threshold.

### SUMMARY

Accordingly, it is an object of one or more embodiments of the present patent application to provide a system for delivering sensory stimulation. The system comprises: a sensor configured to measure autonomic nervous system activity information of a user during a sleep session; a sensory stimulator configured to deliver sensory simulation to the user during the sleep session; and a computer system that comprises one or more physical processors operatively connected with the sensor and the sensory stimulator. The one or more physical processors are programmed with computer program instructions which, when executed cause the computer system to: obtain the autonomic nervous system activity information of the user; determine parasympathetic nervous system information based on the obtained autonomic nervous system activity information of the user; and provide input to the sensory stimulator based on the determined parasympathetic nervous system information, the provided input causing the sensory stimulator to deliver the sensory stimulation to the user based on the determined parasympathetic nervous system information.

It is yet another aspect of one or more embodiments of the present patent application to provide a method for delivering sensory stimulation. The method is implemented by a computer system comprising one or more physical processors executing computer program instructions that, when executed, perform the method. The method comprises obtaining, from a sensor, autonomic nervous system activity information of a user during a sleep session; determining, using the computer system, parasympathetic nervous system information based on the obtained autonomic nervous system activity information of the user; and providing, using the computer system, input to a sensory stimulator (104) based on the determined parasympathetic nervous system information, the provided input causing the sensory stimulator to deliver the sensory stimulation to the user based on the determined parasympathetic nervous system information.

It is yet another aspect of one or more embodiments to provide a system for delivering sensory stimulation. The system comprises a means for measuring autonomic nervous system activity information of a user during a sleep session; a means for delivering sensory stimulation to the user during the sleep session; and a means for executing machine-readable instructions with at least one processor. The machine-readable instructions comprise obtaining, from the means for sensing, autonomic nervous system activity information of the user; determining, using the means for executing, parasympathetic nervous system information based on the obtained autonomic nervous system activity information of the user; providing, using the means for executing, input to the means for delivering based on the determined parasympathetic nervous system information, the provided input causing the means for delivering to deliver the sensory stimulation to the user based on the determined parasympathetic nervous system information.

These and other objects, features, and characteristics of the present patent application, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present patent application.

### BRIEF DESCRIPTION OF THE DRAWINGS

FIG. 1 shows an exemplary system that detects NREM3 through brain activity and starts stimulation through built-in headphones;

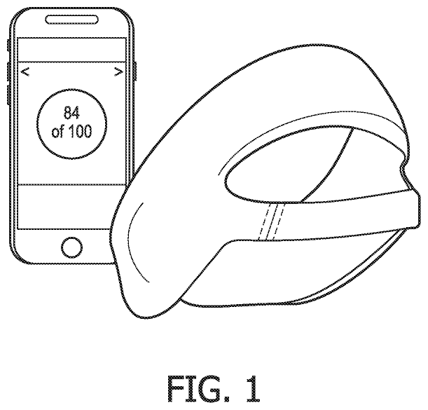

FIG. 2 shows an exemplary system for delivering sensory stimulation in accordance with an embodiment of the present patent application; and

FIG. 3 shows an exemplary method for delivering sensory in accordance with an embodiment of the present patent application.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. As used herein, the term “or” means “and/or” unless the context clearly dictates otherwise.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

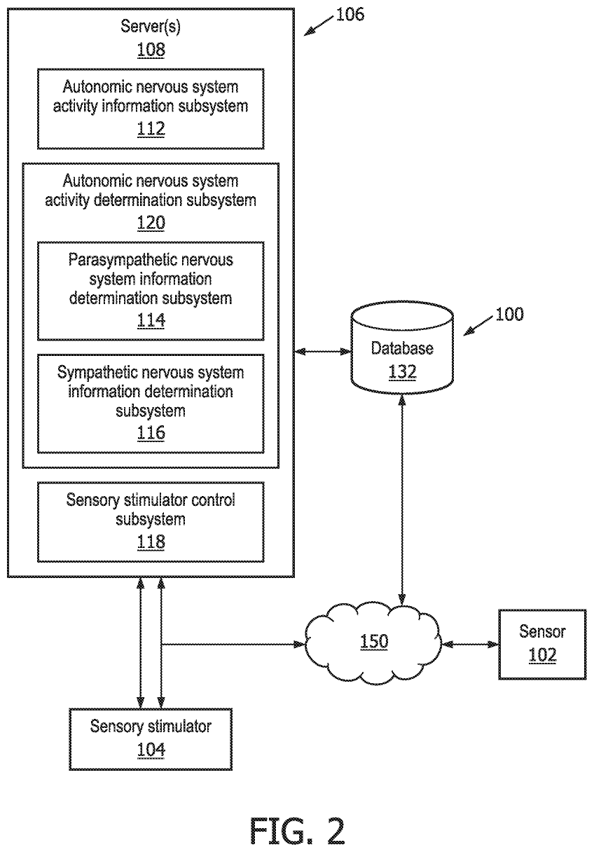

Referring to FIG. 2, the present patent application provides a system 100 for delivering sensory stimulation. System 100 comprises: a sensor 102 configured to measure autonomic nervous system activity information of a user during a sleep session; a sensory stimulator 104 configured to deliver sensory simulation to the user during the sleep session; and a computer system 106 that comprises one or more physical processors 108 operatively connected with sensor 102 and sensory stimulator 104. One or more physical processors 108 are programmed with computer program instructions which, when executed cause the computer system 106 to: obtain the autonomic nervous system activity information of the user; determine parasympathetic nervous system information based on the obtained autonomic nervous system activity information of the user; and provide input to the sensory stimulator based on the determined parasympathetic nervous system information, the provided input causing the sensory stimulator to deliver the sensory stimulation to the user based on the determined parasympathetic nervous system information.

The autonomic nervous system (ANS) is a division of the peripheral nervous system (PNS) and is responsible for the involuntary functions of the human body. The autonomic nervous system (ANS) generally has two divisions a) parasympathetic nervous system (PNS) and b) sympathetic nervous system (SNS).

The SNS is usually associated with and is responsible for the “fight-or-flight” response, to reflect the fact that the human body is highly aroused with/engaged by the external environment. The SNS controls the human body's responses to a perceived threat. The SNS exhibits a stronger tone during wake, REM sleep and very shallow NREM sleep (stage 1, 2, i.e., NREM1 and NREM2).

On the other hand, the PNS is associated with and is responsible for the “rest-and-digest” processes, indicating the opposite state of the human body. The PNS controls homeostasis. The PNS is highly correlated with vigilance (during wake) and arousability (during the night) and becomes progressively stronger in NREM sleep, usually being the strongest in NREM3 (NREM sleep stage 3).

In some embodiments, the present patent application proposes an intervention in which sensory (e.g., auditory) stimulation is delivered to sleeping subjects, users or users to enhance sleep slow waves when the parasympathetic nervous system (PNS) tone of the autonomic nervous system is high. In some embodiments, the benefits of this intervention compared to a system where sensory (e.g., auditory) stimulation is delivered during NREM3 is that (1) the intervention can be considerably more comfortable as markers of the parasympathetic nervous system (PNS) tone can already be acquired from a heart beat signal or other ANS activity signals; (2) it guarantees stimulation during the least arousable state of the user/subject/patient, and (3) it also provides opportunities to stimulate outside NREM3 (NREM sleep stage 3), which becomes important in individuals, subjects, users or patients who experience very little NREM3 (e.g., older individuals, subjects, users or patients). Several embodiments are proposed in the present patent application covering a range of methods with which markers for the parasympathetic nervous system (PNS) tone can be estimated and several stimulation strategies are proposed based on these markers.

In some embodiments, sensor 102 is configured to measure physiological characteristics from which the sympathetic nervous system (SNS) tone and the parasympathetic nervous system (PNS) tone can be quantified. In some embodiments, sensor 102 is any sensor that is configured to measure a physiological modality from which the ANS activity can be quantified. In some embodiments, sensor 102 is configured to generate output signals conveying information related to the ANS activity of the user. In some embodiments, sensor 102 is configured to generate output signals conveying information related to the sympathetic nervous system (SNS) tone and the parasympathetic nervous system (PNS) tone of the user.

As another example, the information related to the ANS activity of the user may be obtained from one or more monitoring devices (e.g., heart rate variability monitoring device, respiratory effort monitoring device, blood pulse reflection monitoring device, blood pressure monitoring device, digestive activity monitoring device, or other ANS activity monitoring devices). In some embodiments, one or more monitoring devices and associated sensors 102 may be configured to monitor the ANS activity of user. These monitoring devices may include one or more sensors 102. Sensor 102 may, for instance, be configured to obtain information related to the ANS activity of user.

In some embodiments, sensor 102 includes a transmitter for sending signals/information and a receiver for receiving the signals/information. In some embodiments, sensor 102 is configured to communicate wirelessly with computer system 106. As shown in FIG. 2, in some embodiments, sensor 102 is configured to be operatively connected with computer system 106 and/or one or more physical processors 108 of computer system 106. In some embodiments, sensor 102 is configured to communicate with sensory stimulator 104. In some embodiments, sensor 102 is in communication with a database 132. In some embodiments, the information related to the ANS activity of the user may be obtained from the database 132 that is being updated in real-time by sensor 102.

In one scenario, a monitoring device may obtain information (e.g., based on information from sensor 102), and provide information to computer system 106 (e.g., comprising server 108) over a network (e.g., network 150) for processing. In another scenario, upon obtaining the information, the monitoring device may process the obtained information, and provide processed information to computer system 106 over a network (e.g., network 150). In yet another scenario, the monitoring device may automatically provide information (e.g., obtained or processed) to computer system 106 (e.g., comprising server 108).

In some embodiments, sensor 102 may include one or more sensors disposed in a plurality of locations, such as for example, within (or in communication with) sensory stimulator 104, coupled (in a removable manner) with clothing of the user, worn by the user (e.g., as a headband, a wristband, a hood worn by the user etc.), positioned to point at the user while the user sleeps (e.g., a camera that conveys output signals related to movement of the user), and/or in other locations near/proximity to the user.

In some embodiments, sensor 102 is worn on the body of the user/user. In some embodiments, sensor 102 is mountable on the wrist or the finger-tip of the user. In some embodiments, sensor 102 is mounted on the chest of the user. In some embodiments, sensor 102 is mounted on belts worn around the thorax or abdomen of the user.

In some embodiments, sensor 102 is positioned below the body of the user (e.g., in the bed or under the mattress, when the user is sleeping). In some embodiments, sensor 102 is not in contact with the user, but, for instance, comprise load or pressure sensors mounted on top of or under the mattress the user lies on during sleep. In some embodiments, sensor 102 (e.g., Doppler radar sensor) is mounted on, for instance, the night table next to the user's bed. In some embodiments, sensor 102 can be completely contact less (e.g., camera-based, Doppler radar or light based) sensor. In some embodiments, sensor 102 comprises one or more light sources for illuminating a part of the user's body and one or several detectors for detecting the light from the user's body.

In some embodiments, sensor 102 may include heart rate variability sensor, respiratory effort sensor, blood pulse wave sensor, blood pressure sensor, digestive activity sensor, or other ANS activity sensor. In some embodiments, sensor 102 includes any combination of the above-noted sensors.

In some embodiments, sensor 102 is configured to sense or measure heart rate variability of the user. In some embodiments, sensor 102 includes a photoplethysmogram sensor, an electrocardiogram sensor, a ballistocardiogram sensor, a seismocardiogram sensor, a radar sensor, a doppler radar sensor, an ultrasound sensor, an echocardiogram sensor, or any other heart rate variability sensors.

In some embodiments, sensor 102 is configured to sense or measure a body temperature of the user. In some embodiments, sensor 102 includes a body temperature sensor.

In some embodiments, sensor 102 is configured to sense or measure respiratory effort of the user. In some embodiments, sensor 102 includes an airflow sensor, a respiratory inductance plethysmogram, RIP sensor , a ballistocardiogram sensor, a radar sensor, a doppler radar sensor, a photoplethysmogram sensor, a thermistor cannula sensor, or any other respiratory effort sensors. In some embodiments, sensor 102 are configured to measure respiratory flow using the thermistor cannula sensors. In some embodiments, the cannula sensors are placed on the nose and/or mouth of the user. In some embodiments, sensor 102 is configured to sense or measure respiration rate variability of the user. In some embodiments, sensor 102 is configured to sense or measure tidal volume of the user.

In some embodiments, sensor 102 is configured to sense or measure blood pulse reflection of the user. In some embodiments, sensor 102 includes a photoplethysmogram sensor, a blood pressure sensor, a tonometry sensor, an ultrasound sensor, or any other blood pulse wave sensors.

In some embodiments, sensor 102 is configured to sense or measure blood pressure of the user. In some embodiments, sensor 102 includes a blood pressure cuff, a tonometry sensor, a pulse arrival time estimator, a pulse wave velocity estimator, a pulse transit time estimator, or any other blood pressure sensors.

In some embodiments, sensor 102 is configured to sense or measure pulse wave velocity of the user. In some embodiments, sensor 102 is configured to sense or measure pulse transit time of the user. In some embodiments, sensor 102 is configured to sense or measure pulse arrival time of the user.

In some embodiments, sensor 102 is configured to sense or measure digestive activity of the user. In some embodiments, sensor 102 is configured to sense or measure digestive activity of the user in the user's gastrointestinal tract or in the user's kidneys. In some embodiments, sensor 102 is configured to sense or measure blood perfusion in the digestive organs (e.g., gastrointestinal tract or kidneys) of the user. In some embodiments, sensor 102 is configured to sense or measure myographic activity in the muscles around the digestive organs (e.g., gastrointestinal tract or kidneys) of the user. In some embodiments, sensor 102 is configured to sense or measure the movement of the contents within the digestive organs (e.g., gastrointestinal tract or kidneys) of the user.

In some embodiments, sensor 102 includes a camera with a light source (e.g., infrared invisible to the human eye). In some embodiments, this camera with the light source may also be referred to as vital signs camera.

In some embodiments, sensory stimulator 104 is also referred to as an actuator or a stimulation actuator. In some embodiments, sensory stimulator 104 is configured to receive information from computer system 106 and to generate and provide sensory stimulation to the user based on the received information.

In some embodiments, the received information from computer system 106 may include information that the PNS tone is high enough to begin the sensory stimulation. In some embodiments, sensory stimulator 104 is configured to generate and provide sensory stimulation to enhance slow waves when the received information from computer system 106 denotes a high parasympathetic nervous system (PNS) tone. In some embodiments, sensory stimulator 104 is configured to adjust the intensity of sensory stimulation provided to the user based on the information received from computer system 106.

In some embodiments, the sensory stimulations include 1) auditory stimulations of which the volume depends on the strength of the PNS tone, 2) other stimulation types where it is paramount that the stimulation is only delivered during a low arousability state.

In some embodiments, the received information from computer system 106 may include information that (e.g., (after starting the sensory stimulation)) the PNS tone is too low to continue stimulation or a sudden burst of the SNS activity indicates one or more arousals, after which the sensory stimulation should cease. In some embodiments, sensory stimulator 104 is configured to stop or start sensory stimulation provided to the user based on the information received from computer system 106.

While in prior art auditory slow wave enhancement is driven by the detection of actual slow waves, the present patent application does not require presence of slow waves (i.e., it's actually incapable of detecting slow waves). In some embodiments, the high PNS tone as the acting trigger in the present patent application might not only be used to deliver a stimulation therapy similar to what is known from the prior art, but also to facilitate faster transitions to NREM3 sleep with the potential to also extending the duration of NREM3 sleep periods.

In some embodiments, sensory stimulator 104 is configured to provide sensory stimulation to the user during the least arousable state of the user based on the received information related to the ANS activity of the user (e.g., the PNS tone is high enough). In some embodiments, sensory stimulator 104 is configured to provide sensory stimulation to the user outside NREM3 (NREM sleep stage 3), which becomes important in individuals or users who experience very little NREM3 (e.g., older individuals or users), based on the received information related to the ANS activity of the user (e.g., the PNS tone is high enough).

In some embodiments, sensory stimulator 104 is configured to provide sensory stimulation to the user prior to a sleep session, during a sleep session, after a sleep session, and/or at other times. In some embodiments, sensory stimulator 104 is configured to provide sensory stimulation to the user without causing arousals during sleep. In some embodiments, for example, sensory stimulator 104 may be configured to provide sensory stimulation to the user during slow wave sleep in the sleep session. In some embodiments, sensory stimulator 104 may be configured to provide sensory stimulation to the user to induce and/or adjust slow wave activity (SWA) in the user. In some embodiments, sleep slow waves are associated with slow wave activity (SWA) in the user during the sleep session. In some embodiments, sensory stimulator 104 is configured such that inducing and/or adjusting SWA includes inducing, increasing, and/or enhancing sleep slow waves in the user.

In some embodiments, sensory stimulator 104 may be configured to induce, increase, and/or enhance sleep slow waves through non-invasive brain stimulation and/or other methods. In some embodiments, sensory stimulator 104 may be configured to induce, increase, and/or enhance sleep slow waves through non-invasive brain stimulation using sensory stimulation.

In some embodiments, a sensory stimulation is performed by sensory stimulator 104 based on the activity of the autonomic nervous system (ANS). In some embodiments, the sensory stimulation may include different types of sensory stimulations. In some embodiments, the sensory stimulation is selected from the group consisting of olfactory stimulation, somatosensory stimulation, auditory stimulation, visual stimulation, touch stimulation, taste stimulation, haptic stimulation, peripheral stimulation, transcranial stimulation, transcranial magnetic stimulation. electric stimulation and magnetic stimulation. In some embodiments, the sensory stimulation may include smells, tones, odors, sounds, visual stimulation (e.g., lights flashed on open and/or closed eyes), touches, tastes, haptic (e.g., vibrations or non-contact haptic) stimulation, and/or other sensory stimulations. In some embodiments, for example, acoustic tones may be provided to the user to induce, increase, and/or enhance sleep slow waves. In some embodiments, examples of sensory stimulator 104 may include one or more of a music player, a tone generator, a collection of electrodes on the scalp of the user, a unit to deliver vibratory stimulation (also known as somato-sensory stimulation), a coil generating a magnetic field to directly stimulate the brain's cortex, light generators, a fragrance dispenser, and/or other sensory stimulators.

In some embodiments, sensory stimulator 104 includes wireless audio device and one or more audio speakers. In some embodiments, a headband may be worn by the user. In some embodiments, the headband includes the wireless audio device and the one or more audio speakers. In some embodiments, the one or more audio speakers may be located in and/or near the ears of the user.

In some embodiments, interchangeable earbud attachments in a range of sizes (e.g., small (S), medium (M), and large (L) are configured to removably couple with a housing of ear insert such that the user may find an interchangeable earbud attachment that is most comfortable and attach it to the housing. In some embodiments, earbud attachments are formed from the conductive materials such that earbud attachments form a portion of sensor 102.

In some embodiments, the housing includes electronic components that form a portion of sensor 102, sensory stimulator 104, and/or other components of system 100. In some embodiments, sensory stimulator 104, sensor 102 are formed integrally with the ear insert such that the ear insert, sensory stimulators 104, and sensor 102 appear to form a single unified physical object that is comfortable for the user to insert and remove from his or her ears. In some embodiments, the ear insert is custom formed based on three dimensional data representative of the ear of the user, a physical mold/model of the ear of the user, and/or other information. Whether a custom ear insert is fabricated directly based on the three dimensional data or fabricated from a mold or other components formed based on the three dimensional data, the ear insert may be customized based on the three dimensional data to fill the ear canal of the user and much of the outer ear of the user without protruding from the head of the user so as to enable comfort during sleep and/or other activities (e.g., maximizing the surface area ear insert that is in contact with the ear of the user without hindering comfort). In some embodiments, the ear insert may be customized to include a small canal built into the ear insert (e.g., with an approximate diameter of about 2-3 mm) to facilitate hearing ambient sounds (e.g., during sleep and/or while awake). In some embodiments, the ear insert may be customized such than an area of the ear insert under the tragus of the user is smoothed to allow the tragus room for lying against the earpiece should the user elect to sleep on their side, for example.

In some embodiments, the housing and the earbud attachments form correspond clamping or other engagement surfaces configured to removably couple with each other. In some embodiments, engagement surface on the housing includes a conductive surface such that electrical signals passing through the earbud attachments are received by the housing. In some embodiments, the housing may include one or more features (e.g., a hook that wraps around the ear of the user) to enhance coupling with the ear of the user. In some embodiments, the ear insert are made at three different levels of material elasticity (e.g., very soft, soft, and hard).

In some embodiments, system 100 includes computer system 106 that comprises one or more physical processors 108 operatively connected with sensor 102 and sensory stimulator 104. In some embodiments, one or more physical processors 108 are programmed with computer program instructions which, when executed cause computer system 106 to perform various functions.

In some embodiments, computer system 106 is configured to extract features or characteristics proportional to the tone of each of the autonomic divisions (i.e., the SNS and the PNS); and c) computer system 106 is also configured to, based on the features extracted by the computer system 106, decide when: 1) the PNS tone is high enough to begin the sensory stimulation or 2) (after starting the sensory stimulation) when the PNS tone is too low to continue stimulation or a sudden burst of the SNS activity indicates one or more arousals, after which the sensory stimulation should cease.

In some embodiments, computer system 106 is configured to monitor the balance between the sympathetic nervous system (SNS) and the parasympathetic nervous system (PNS). In some embodiments, a high PNS is determined to be a better queue for the sensory stimulation, as it is associated with a higher arousal threshold (and a lower arousability) of the user. It is known that the PNS tone can already become high in NREM2, which would provide a suitable stimulation opportunity in NREM2, therefore being more suitable for older aged individuals, patients or users.

As shown in FIG. 2, system 100 may comprise server 108 (or multiple servers 108). In some embodiments, server 108 includes one or more physical/hardware processors 108. In FIG. 2, database 132 is shown as a separate entity, but, in some embodiments, database 132 could be part of computer system 106.

In some embodiments, server 108 comprises autonomic nervous system activity information subsystem 112, autonomic nervous system activity determination subsystem 120, parasympathetic nervous system information determination subsystem 114, sympathetic nervous system information determination subsystem 116, sensory stimulator control subsystem 118 or other components or subsystems.

As will be clear from the discussions above and below, in some embodiments, system 100 includes computer system 106 that has one or more physical/hardware processors 108 programmed with computer program/machine readable instructions that, when executed cause computer system 106 to obtain information or data from sensor 102. In some embodiments, computer system 106 may also be referred to as means 106 for executing machine readable instructions with at least one hardware processor 108.

In some embodiments, autonomic nervous system activity information subsystem 112 is configured to receive or obtain the autonomic nervous system activity information of the user from sensor 102. In some embodiments, the autonomic nervous system activity information may include heart rate variability information, respiratory effort information, tidal volume information, respiratory rate variability information, blood pulse reflection information, blood pressure information, digestive activity information, airflow information, initial aortic blood pulse information, reflected blood pulses information in systolic and diastolic phases, blood perfusion information in the digestive organ (e.g., gastrointestinal tract or kidneys), myographic activity level information in the muscles around the digestive organ, information about the movement of the contents within these digestive organs, pulse transit time information, pulse wave velocity information, pulse arrival time information, or other autonomic nervous system activity related information.

In some embodiments, autonomic nervous system activity information subsystem 112 is configured to further process the received or obtained autonomic nervous system activity information. In some embodiments, autonomic nervous system activity information subsystem 112 comprises an ANS activity signal feature extraction unit that is configured to extract the ANS activity features from the signals, data or information provided by sensor 102.

In some embodiments, autonomic nervous system activity information subsystem 112 is optional and the autonomic nervous system activity information of the user from sensor 102 may be directly received or obtained by autonomic nervous system activity determination subsystem 120. In such an embodiment, autonomic nervous system activity determination subsystem 120 comprises an ANS activity feature extraction unit that is configured to extract the ANS activity features from the signals, data or information provided by sensor 102.

In some embodiments, autonomic nervous system activity determination subsystem 120 is configured to extract a characteristic from the signals or information obtained from sensor 102 and that is proportional to the ANS activity. In some embodiments, autonomic nervous system activity determination subsystem 120 includes extraction algorithms configured to extract a characteristic from the signals or information obtained from sensor 102 and that is proportional to the ANS activity. In some embodiments, autonomic nervous system activity determination subsystem 120 includes machine learning based methods (i.e., that were trained using historical data/information) are configured to extract a characteristic from the signals or information obtained from sensor 102 and that is proportional to the ANS activity. In some embodiments, autonomic nervous system activity determination subsystem 120 may include a processing module or a signal processing module.

In some embodiments, autonomic nervous system activity determination subsystem 120 includes parasympathetic nervous system information determination subsystem 114 and sympathetic nervous system information determination subsystem 116. In some embodiments, system 100 does not include parasympathetic nervous system information determination subsystem 114 and sympathetic nervous system information determination subsystem 116 and system 100 only includes autonomic nervous system activity determination subsystem 120 that is configured to perform all the functions of parasympathetic nervous system information determination subsystem 114 and sympathetic nervous system information determination subsystem 116. In such an embodiment, autonomic nervous system activity determination subsystem 120 is configured to determine both the parasympathetic nervous system information and sympathetic nervous system information.

In some embodiments, system 100 does not include sympathetic nervous system information determination subsystem 116 and system 100 only includes autonomic nervous system activity determination subsystem 120 and parasympathetic nervous system information determination subsystem 114. In such an embodiment, autonomic nervous system activity determination subsystem 120 and parasympathetic nervous system information determination subsystem 114 are configured to determine both the parasympathetic nervous system information and sympathetic nervous system information.

In some embodiments, system 100 does not include parasympathetic nervous system information determination subsystem 114 and system 100 includes only autonomic nervous system activity determination subsystem 120 and sympathetic nervous system information determination subsystem 116. In such an embodiment, autonomic nervous system activity determination subsystem 120 and sympathetic nervous system information determination subsystem 116 are configured to determine both the parasympathetic nervous system information and sympathetic nervous system information.

In some embodiments, autonomic nervous system activity determination subsystem 120 is configured to extract a characteristic that is proportional to the ANS activity from the heart rate variability information obtained from heart rate variability sensor 102. In some embodiments, autonomic nervous system activity determination subsystem 120 is configured to process a statistical property of the heart rate variability in the time domain, frequency domain or non-linear domain.

In some embodiments, system 100 of the present patent application is configured to use the ANS balance for the sensory stimulation. In some embodiments, one of the most commonly used surrogates for measuring the ANS activity is based on the analysis of cardiac signals: namely the heart rate variability (HRV). There are distinct patterns in the HRV that become more and more pronounced as the PNS tone increases. These patterns can be quantified through, for example, frequency analysis, fractal analysis, fluctuation analysis or entropy analysis. These types of quantifications could be used to steer the sensory stimulation. The extent to which the patterns are present could be used to modulate the level of the sensory stimulation, meaning that the sensory stimulation could be permitted to be louder when the user is less likely to be aroused by audio.

In some embodiments, time domain features of heart rate variability include means and medians of heart rate and inter-beat (RR) interval (both detrended and absolute). In some embodiments, time domain features of heart rate variability include standard deviation of the RR interval, percentage of successive the RR interval differences >50 millisecond (ms); root mean square of successive the RR interval differences, standard deviation of successive the RR interval differences; mean absolute difference (both detrended and absolute RR). In some embodiments, time domain features of heart rate variability include percentiles (5%, 10%, 25%, 50%, 75%, 90% and 95%) of detrended and absolute heart rate and inter-beat (RR) interval. In some embodiments, time domain features of heart rate variability include the RR interval detrended fluctuation analysis (DFA), its short, long exponents and all scales, and windowed DFA over 330 seconds and progressive DFA over non-overlapping segments of 64 heartbeats.

In some embodiments, frequency domain features of heart rate variability include RR logarithmic very low frequency, low frequency, and high frequency power and low frequency-to-high frequency ratio on 270 seconds windows. In some embodiments, frequency domain features of heart rate variability include Boundary-adapted RR logarithmic very low frequency, low frequency, and high frequency power and low frequency-to-high frequency ratio on 270 seconds windows. In some embodiments, frequency domain features of heart rate variability include RR mean respiratory frequency and power, max phase and module in high frequency pole.

In some embodiments, entropy and regularity features of heart rate variability include Multiscale sample entropy 1 of RR intervals at length 1 and 2, scales 1-10 over 510 seconds. In some embodiments, entropy and regularity features of heart rate variability include sample entropy of symbolic binary changes in RR intervals. In some embodiments, short- and long-range phase coordination of R-R intervals in patterns of up to 8 consecutive heartbeats. In some embodiments, entropy and regularity features of heart rate variability include phase synchronization for 6:2, 7:2, 8:2 and 9:2 phases, dominant ratio, short- and long-term coordination. In some embodiments, entropy and regularity features of heart rate variability include Higuchi's fractal dimension of the normalized IBI sequence. In some embodiments, inter-beat-interval (i.e., the time difference between two heart beats). In some embodiment, obtaining this for a series of heart beats gives you an IBI sequence.

In some embodiments, miscellaneous features of heart rate variability include mean teager energy, % of transition points and maxima and mean and standard deviation of intervals between them, mean and standard deviation of the amplitude of normalized IBIs at transition points and maxima. In some embodiments, miscellaneous features of heart rate variability include arousal probabilities (maximum, mean, median, minimum, standard deviation). In some embodiments, miscellaneous features of heart rate variability include visibility graph features.

In some embodiments, autonomic nervous system activity determination subsystem 120 is configured to extract a characteristic that is proportional to the ANS activity from the respiratory effort information obtained from respiratory effort sensor 102. In some embodiments, autonomic nervous system activity determination subsystem 120 is configured to process respiration rate variability or the tidal volume.

In some embodiments, autonomic nervous system activity determination subsystem 120 is configured to extract a characteristic that is proportional to the ANS activity from the blood pulse reflection information obtained from blood pulse wave sensor 102. In some embodiments, autonomic nervous system activity determination subsystem 120 is configured to process the relative proportion or timing between the initial aortic blood pulse and the reflected blood pulses in the systolic and diastolic phase.

In some embodiments, autonomic nervous system activity determination subsystem 120 is configured to extract a characteristic that is proportional to the ANS activity from the blood pressure information obtained from blood pressure sensor 102. In some embodiments, autonomic nervous system activity determination subsystem 120 is configured to process the value of the blood pressure which directly related to ANS activity.

In some embodiments, autonomic nervous system activity determination subsystem 120 is configured to extract a characteristic that is proportional to the ANS activity from the digestive activity information obtained from digestive activity sensor 102. In some embodiments, autonomic nervous system activity determination subsystem 120 is configured to process a quantification of blood perfusion in the digestive organ, or myographic activity level in the muscles around the digestive organ, or the movement of the contents within these digestive organs. In some embodiments, the digestive organs include gastrointestinal tract or kidneys.

In some embodiments, parasympathetic nervous system information subsystem 114 is configured to determine parasympathetic nervous system information based on the obtained autonomic nervous system activity information of the user.

In some embodiments, parasympathetic nervous system information may include information that indicates reduced heart rate, constricted bladder, erection of genitals of man and woman, stimulated activity of gall bladder, stimulated activity of pancreas, stimulated activity of the digestive organs (e.g., gastrointestinal tract or kidneys), constricted bronchia, increased saliva production, constricted pupils, or other parasympathetic nervous system related information.

In some embodiments, NREM3 (NREM third stage/deepest Non-Rapid Eye Movement) sleep is detected using cardiorespiratory signals from sensor 102. The cardiorespiratory system during sleep enters a distinct state during NREM3. This can be detected using a heartbeat sensor 102 (e.g., electrocardiographic (ECG) sensor) and a respiratory effort sensor 102 (e.g., respiratory inductance plethysmogram, RIP). These sensors 102 are less obtrusive than a head-worn cap. There are alternative ways to measure these cardiorespiratory signals that are even less obtrusive, for example, 1) a photoplethysmographic (PPG) sensor 102 in a wrist watch (that does not require sticky electrodes, unlike ECG or EEG electrodes) of the patient/user, 2) a ballistocardiographic sensor 102 in the bed (that does not require to be attached to the body of the patient/user), 3) a radio frequency sensor 102 on a night stand (that does not require to be in contact with the patient/user), and 4) a camera 102 with a light source (e.g., infrared invisible to the human eye) for remote registration of plethysmogram sensor 102 on a night stand.

In some embodiments, autonomic nervous system activity determination subsystem 120 or parasympathetic nervous system information subsystem 114 is configured to determine if the parasympathetic nervous system information falls within a predetermined range to begin sensory stimulation. In some embodiments, autonomic nervous system activity determination subsystem 120 or parasympathetic nervous system information subsystem 114 is configured to determine if the parasympathetic nervous system information falls outside the predetermined range to stop sensory stimulation. In some embodiments, the predetermined range include a PNS range in which the PNS tones or values are high. In some embodiments, a measure of PNS ranges between 0 and 1. In some embodiments, there are a few ways to do this: a) pick a point (e.g., 0.5) to use. b) the point can be then optimized based on user tests (i.e., picking a point high enough to not wake people up, but still with enough coverage throughout the night to play tones). In some embodiments, the volume of the tone can made dependent on the PNS value. In some embodiments, if the PNS value is 1, then the tone is provided/played at max volume (e.g., which is pre-determined in user tests).

In some embodiments, for values below the max volume, {pns tone}*{max volume}*a+b where a and b can be pre-determined constants that results in an ideal PNS-to-volume mapping can be applied. In some embodiments, many of these parameters can be learnt in an online fashion (i.e. during usage). In some embodiments, for example, some average values that work well for most people could be used as a starting point, but when the system senses that it is waking up the person too much, the values can automatically adapt to a more conservative tone playing strategy. In some embodiments, the above can be done, for example, through reinforcement learning, in which an artificial agent tries out different tone delivery strategies, observes the effect, and improves its own strategy over time.

In some embodiments, autonomic nervous system activity determination subsystem 120 or parasympathetic nervous system information subsystem 114 is configured to determine if the parasympathetic nervous system information is above a predetermined threshold to begin sensory stimulation. In some embodiments, autonomic nervous system activity determination subsystem 120 or parasympathetic nervous system information subsystem 114 is configured to determine if the parasympathetic nervous system information is below the predetermined threshold to stop sensory stimulation. In some embodiments, the predetermined threshold includes a PNS tone or value above which the PNS tones or values are high.

In some embodiments, autonomic nervous system activity determination subsystem 120 or parasympathetic nervous system information determination subsystem 114 is also configured to detect a decrease of the PNS activity below a given threshold. In some embodiments, after this point, the arousal threshold of the user probably decreases (i.e., his/her arousability increases) and from this point on (until the PNS tone or value increases again, later during the night), sensory stimulation will probably lead to an arousal or an awakening, or simply not be altogether effective.

In some embodiments, autonomic nervous system activity determination subsystem 120 or parasympathetic nervous system information determination subsystem 114 is configured to provide input to sensory stimulator 104 based on the detected/determined decrease in the parasympathetic nervous system information/PNS activity, the provided input causing sensory stimulator 104 to stop delivering the sensory stimulation to the user based on the detected/determined decrease in the parasympathetic nervous system information/ PNS activity.

In some embodiments, sympathetic nervous system information subsystem 116 is configured to determine sympathetic nervous system information based on the obtained autonomic nervous system activity information of the user.

In some embodiments, sympathetic nervous system information may include information that indicates increased heart rate, relaxed bladder, stimulated orgasms of genitals of man and woman, stimulated adrenal medulla to release adrenaline and noradrenaline, inhibited activity of the gall bladder, inhibited activity of pancreas, inhibited activity of the digestive organs (e.g., gastrointestinal tract or kidneys), dilated bronchia, inhibited saliva production, dilated pupils, or other , parasympathetic nervous system related information.

In some embodiments, autonomic nervous system activity determination subsystem 120 or sympathetic nervous system information subsystem 116 is configured to determine if the sympathetic nervous system information falls within a predetermined range to stop sensory stimulation. In some embodiments, autonomic nervous system activity determination subsystem 120 or sympathetic nervous system information subsystem 116 is configured to determine if the sympathetic nervous system information falls outside the predetermined range to start sensory stimulation. In some embodiments, the predetermined range include a SNS range in which the SNS tones or values are high. In some embodiments, the description for the SNS tone is analogous to the above description for PNS tone.

In some embodiments, autonomic nervous system activity determination subsystem 120 or sympathetic nervous system information subsystem 116 is configured to determine if the sympathetic nervous system information is below a predetermined threshold to stop sensory stimulation. In some embodiments, autonomic nervous system activity determination subsystem 120 or sympathetic nervous system information subsystem 116 is configured to determine if the sympathetic nervous system information is above the predetermined threshold to start sensory stimulation. In some embodiments, the predetermined threshold includes a SNS tone or value below which the SNS tones or values are high. In some embodiments, the predetermined threshold may be referred to as arousal threshold.

In some embodiments, autonomic nervous system activity determination subsystem 120 or sympathetic nervous system information determination subsystem 116 is configured to detect the occurrence of an autonomic arousal; these are associated with burst in the SNS activity, which have very specific hallmarks such as increase in heart rate and blood pressure, body movements, abrupt changes in breathing frequency or breathing pattern often associated with a sudden increase in tidal volume, etc. In some embodiments, these changes can be usually detected with the same input signals or information from sensor 102 as described before.

In some embodiments, autonomic nervous system activity determination subsystem 120 or sympathetic nervous system information determination subsystem 116 is configured to provide input to sensory stimulator 104 based on the detected/determined increase in the sympathetic nervous system information/SNS activity, the provided input causing sensory stimulator 104 to stop delivering the sensory stimulation to the user based on the detected/determined increase in the sympathetic nervous system information/SNS activity.

In order to prevent awakenings, sensory stimulator 104 is configured to stop stimulation when the PNS activity decreases. In order to prevent awakenings, sensory stimulator 104 is configured to stop stimulation when the SNS activity increases. For example, this could be due to an arousal (e.g., endemic, or caused by the actual stimulation), or due to a natural sleep progression, where after a period of NREM3 sleep, the sleep cycle continues, often followed by NREM2 and eventually REM sleep.

In some embodiments, as an alternative to stopping the stimulation immediately upon an arousal, system 100 is also configured to continue the stimulation and monitor for the occurrence of more arousals. This is due to the fact that some arousals are not caused by the stimulation itself but by other (e.g., cortical or autonomic) phenomena and, therefore, may not be relevant in regard to the stimulation strategy. If after the first arousal, the stimulation is continued and no other arousal occurred, then it is safe to continue the stimulation. If more arousals followed the first arousal, then system 100 is configured to stop the stimulation.

In some embodiments, autonomic nervous system activity determination subsystem 120 is configured to detect a possible arousal event in the user based on the obtained autonomic nervous system activity information. In some embodiments, autonomic nervous system activity determination subsystem 120 is configured to detect possible arousal events, and/or the likelihood of producing arousals based on the obtained autonomic nervous system activity information, and/or by other methods. An arousal event may include waking from sleep and/or other arousal events associated with wakefulness of the user. Responsive to detecting the possible arousal event, sensory stimulator control subsystem 118 may cause sensory stimulator 104 to cease providing sensory stimulation and then determine whether the possible arousal event was a false arousal event. In some embodiments, responsive to determining that the possible arousal event was a false arousal event, sensory stimulator control subsystem 118 may cause sensory stimulator 104 to resume/continue providing sensory stimulation with an intensity determined based on the recently determined parasympathetic nervous system information.

In some embodiments, sensory stimulator control subsystem 118 is configured to control sensory stimulator 104 such that a volume of auditory stimulation (for example) is first provided at a level that is approximately equal to a hearing threshold (e.g., subjectively determined via a calibration during wakefulness) of the user and then progressively adjusted (e.g., increased) as described above (based on the recently determined parasympathetic nervous system information). In some embodiments, an upper limit for the intensity of the sensory stimulation (e.g., volume) may also be subjectively determined during a previous calibration.

If a possible arousal is detected by system 100 during sensory stimulation, sensory stimulator control subsystem 118 controls sensory stimulator 104 such that the stimulation stops. If an arousal is detected outside the sensory stimulation period, the onset of the stimulation is delayed. If no arousal is detected, then system 100 delivers sensory stimulation based on the recently determined parasympathetic nervous system information. As described above, responsive to determining that the possible arousal event was a false arousal event, sensory stimulator control subsystem 118 may cause sensory stimulator 104 to resume/continue providing sensory stimulation with an intensity determined based on the recently determined parasympathetic nervous system information. In some embodiments, system 100 does not automatically revert back to the lowest intensity level in such situations.

In some embodiments, computer system 106 is configured to adjust, in real-time or near real-time, the stimulation intensity (e.g., volume) of the sensory stimulation to enhance the sleep slow waves without provoking arousals in the user.

In some embodiments, sensory stimulator control subsystem 118 is configured to receive parasympathetic nervous system information (from parasympathetic nervous system information determination subsystem 114 or autonomic nervous system activity determination subsystem 120) and to provide input to sensory stimulator 104 based on the determined parasympathetic nervous system information, the provided input causing sensory stimulator 104 to deliver the sensory stimulation to the user based on the determined parasympathetic nervous system information. In some embodiments, the provided input includes starting, continuing, adjusting or stopping the sensory stimulation.

In some embodiments, sensory stimulator control subsystem 118 is configured to receive sympathetic nervous system information (from sympathetic nervous system information determination subsystem 116 or autonomic nervous system activity determination subsystem 120) and to provide input to sensory stimulator 104 based on the determined parasympathetic nervous system information, the provided input causing sensory stimulator 104 to deliver the sensory stimulation to the user based on the determined parasympathetic nervous system information. In some embodiments, the provided input includes starting, continuing, adjusting or stopping the sensory stimulation.

It is known that the expression of heart rate variability (HRV) characteristics may vary heavily from person to person due not only to age-specific differences, but also to the occurrence of specific events or states during daytime, such as stress, level of physical activity, etc.

In some embodiments, system 100 is configured to obtain input parameters from the user. In some embodiments, these input parameters may include age (e.g., by means of a one-time questionnaire where the user could input his/her date of birth), mental states (e.g., subjective impression of current/past level of stress), or the result of daytime activity tracking (e.g., the amount and intensity of physical activity).

In some embodiments, the input parameters are input by the user (or caregiver) into computer system 106 using a user interface. In some embodiments, computer system 106 may include user input parameter information subsystem for further processing this information. In some embodiments, autonomic nervous system activity determination subsystem 120, sympathetic nervous system information determination subsystem 114 or sympathetic nervous system information determination subsystem 116 is configured to receive user input parameter information to determine sympathetic nervous system information or parasympathetic nervous system information using the user input parameter information.

In some embodiments, the provided input causes sensory stimulator 104 to deliver the sensory stimulation to the user when the determined parasympathetic nervous system information is above a predetermined threshold. In some embodiments, system 100 is configured to obtain user input parameter information and determine the predetermined threshold based on the obtained user input parameter information.

In some embodiments, system 100 is configured to adapt some of its system parameters based on the obtain input parameters from the user. In some embodiments, the system parameters include the threshold from which stimulation is done, the sensitivity to arousals upon which stimulation should stop, the intensity of the tones delivered, etc.

In some embodiments, a subsystem of system 100 is configured to continuously obtain subsequent autonomic nervous system activity information. As an example, the subsequent information may comprise additional information corresponding to a subsequent time (after a time corresponding to information that was used to determine input to the sensory stimulator based on the determined parasympathetic nervous system information). The subsequent information may be utilized to further update or modify the ranges and thresholds for the PNS and SNS information of the user (e.g., new information may be used to dynamically update or modify the ranges and thresholds for the PNS and SNS information of the user), etc. For example, the subsequent information may also be configured to provide further input to determine the ranges and thresholds for the PNS and SNS information of the user. In some embodiments, a subsystem of system 100 may be configured to determine the ranges and thresholds for the PNS and SNS information of the user and/or to control sensory stimulator 104 to adjust the sensory stimulation to the user in accordance with a recursively refined profile (e.g., refined through recursive application of profile refinement algorithms) based on previously collected or subsequent autonomic nervous system activity information.

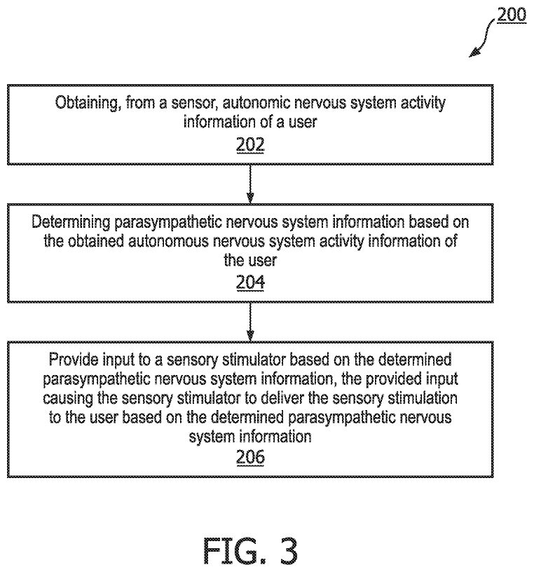

Referring to FIG. 3, a method 200 for delivering sensory stimulation is provided. Method 200 is implemented by computer system 106 that comprises one or more physical/hardware processors 108 executing computer program/machine readable instructions that, when executed, perform method 200. In some embodiments, method 200 comprises obtaining, from sensor 102, the autonomic nervous system activity information of a user at procedure 202; determining parasympathetic nervous system information based on the obtained autonomic nervous system activity information of the user at procedure 204; providing input to sensory stimulator 104 based on the determined parasympathetic nervous system information, the provided input causing sensory stimulator 104 to deliver the sensory stimulation to the user based on the determined parasympathetic nervous system information at procedure 206.

In some embodiments, system 100 may be used with any sleep related products or systems to provide real-time sensory actuation functionality. In some embodiments, system 100 may be used to build sleep slow wave enhancement into a wake-up light or a CPAP therapy device (e.g., sensing in the mask).

In some embodiments, the various computers and subsystems illustrated in FIG. 2 may comprise one or more computing devices that are programmed to perform the functions described herein. The computing devices may include one or more electronic storages (e.g., database 132, or other electronic storages), one or more physical processors programmed with one or more computer program instructions, and/or other components. The computing devices may include communication lines or ports to enable the exchange of information with a network (e.g., network 150) or other computing platforms via wired or wireless techniques (e.g., Ethernet, fiber optics, coaxial cable, WiFi, Bluetooth, near field communication, or other communication technologies). The computing devices may include a plurality of hardware, software, and/or firmware components operating together to provide the functionality attributed herein to the servers. For example, the computing devices may be implemented by a cloud of computing platforms operating together as the computing devices.

The electronic storages may comprise non-transitory storage media that electronically stores information. The electronic storage media of the electronic storages may include one or both of system storage that is provided integrally (e.g., substantially non-removable) with the servers or removable storage that is removably connectable to the servers via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storages may store software algorithms, information determined by the processors, information received from the servers, information received from client computing platforms, or other information that enables the servers to function as described herein.

The processors may be programmed to provide information processing capabilities in the servers. As such, the processors may include one or more of a digital processor, an analog processor, or a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112-120 or other subsystems. The processors may be programmed to execute computer program instructions by software; hardware; firmware; some combination of software, hardware, or firmware; and/or other mechanisms for configuring processing capabilities on the processors. In some embodiments, hardware processors may be interchangeably referred to as physical processors. In some embodiments, machine readable instructions may be interchangeably referred to as computer program instructions.

It should be appreciated that the description of the functionality provided by the different subsystems 112-120 described herein is for illustrative purposes, and is not intended to be limiting, as any of subsystems 112-120 may provide more or less functionality than is described. For example, one or more of subsystems 112-120 may be eliminated, and some or all of its functionality may be provided by other ones of subsystems 112-120. As another example, additional subsystems may be programmed to perform some or all of the functionality attributed herein to one of subsystems 112-120.

It should be appreciated that the different subsystems 112-120 performing the operations illustrated in FIG. 2 may reside in a system with sensor 102 and sensory stimulator 104. In some embodiments, the different subsystems 112-120 performing the operations illustrated in FIG. 2 may reside in an independent monitoring device.

In some embodiments, computer system 106 and/or physical processors/server 108 are included in a smartphone associated with the user and/or other computing devices. In some embodiments, computer system 106 and/or physical processors/server 108 are included in a tablet computer, a laptop computer, a desktop computer, a server computer, and/or other computing devices. In some embodiments, the smartphone comprises an input configured to receive the information in the output signals generated by sensor 102 and/or other information. The input device may be and/or include a microphone included in the smartphone, a USB input device, an Apple Lightning type connector (which can also provide power), a combined microphone/earphones jack, and/or other devices. In some embodiments, converter devices are configured to convert the output signals, and/or the information in the output signals from sensor 102 for transmission to, and receipt by, the smartphone input device. In some embodiments, computer system 106 and/or physical processors/server 108 are configured such that subsystems 112-120, and/or other subsystems form an electronic application (an “app”) running on computer system 106 and/or physical processors/server 108. In some embodiments, the app (as described above related to subsystems 112-120, and/or other subsystems) configured to analyzes the received signal/data/information to determine the information described above.

In some embodiments, system 100 may include a user interface may be configured to provide an interface between system 100 and a user (e.g., a user or a caregiver, etc.) through which the user can provide information to and receive information from system 100. This enables data, results, and/or instructions and any other communicable items, collectively referred to as “information,” to be communicated between the user and system 100. Examples of interface devices suitable for inclusion in user interface include a keypad, buttons, switches, a keyboard, knobs, levers, a display screen, a touch screen, speakers, a microphone, an indicator light, an audible alarm, and a printer. In some embodiments, information may be provided to the user by the user interface in the form of auditory signals, visual signals, tactile signals, and/or other sensory signals. It is to be understood that other communication techniques, either hard-wired or wireless, are also contemplated herein as the user interface. For example, in some embodiments, the user interface may be integrated with a removable storage interface provided by electronic storage 132. In this example, information is loaded into system 100 from removable storage (e.g., a smart card, a flash drive, a removable disk, etc.) that enables the user(s) to customize system 100. Other exemplary input devices and techniques adapted for use with system 100 as user interface include, but are not limited to, an RS-232 port, RF link, an IR link, modem (telephone, cable, Ethernet, internet or other). In short, any technique for communicating information with system 100 is contemplated as the user interface.

In some embodiments, system 100 may also include a communication interface that is configured to send input/control signals to sensory stimulator 104 based on the determined parasympathetic nervous system information through an appropriate wireless communication method (e.g., Wi-Fi, Bluetooth, internet, etc.). In some embodiments, system 100 may include a recursive tuning subsystem that is configured to recursively tune its intelligent decision making subsystem using available data or information to provide better overall adjustment of sensory stimulator 104 and/or better overall control of sensory stimulator 104. In some embodiments, intelligent decision making subsystem, communication interface and recursive tuning subsystem may be part of computer system 106 (comprising server 108).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the present patent application has been described in detail for the purpose of illustration based on what is currently considered to be the most practical and preferred embodiments, it is to be understood that such detail is solely for that purpose and that the present patent application is not limited to the disclosed embodiments, but, on the contrary, is intended to cover modifications and equivalent arrangements that are within the spirit and scope of the appended claims. For example, it is to be understood that the present patent application contemplates that, to the extent possible, one or more features of any embodiment can be combined with one or more features of any other embodiment.

What is claimed is:

1. A system for delivering sensory stimulation, comprising:
a sensor configured to measure autonomic nervous system activity information of a user during a sleep session;
a sensory stimulator configured to deliver sensory stimulation to the user during the sleep session; and
a computer system that comprises one or more physical processors operatively connected with the sensor and the sensory stimulator, the one or more physical processors being programmed with computer program instructions which, when executed cause the computer system to:
obtain the autonomic nervous system activity information of the user;
determine parasympathetic nervous system information based on the obtained autonomic nervous system activity information of the user; and
provide input to the sensory stimulator based on the determined parasympathetic nervous system information, the provided input causing the sensory stimulator to deliver the sensory stimulation to the user based on the determined parasympathetic nervous system information.

2. The system of claim 1, wherein the sensor is selected from the group consisting of a heart rate variability sensor, a respiratory effort sensor, a blood pulse wave sensor, a blood pressure sensor, a body temperature sensor, and a digestive activity sensor.

3. The system of claim 1, wherein the provided input causes the sensory stimulator to deliver the sensory stimulation to the user when the determined parasympathetic nervous system information is above a predetermined threshold, and
wherein the computer system is configured to obtain user input parameter information and to determine the predetermined threshold based on the user input parameter information.

4. The system of claim 1, wherein the sensory stimulation is selected from the group consisting of olfactory stimulation, auditory stimulation, visual stimulation, touch stimulation, taste stimulation, haptic stimulation, peripheral stimulation, transcranial stimulation, transcranial magnetic stimulation. electric stimulation and magnetic stimulation.

5. The system of claim 1, wherein the computer system is configured to determine sympathetic nervous system information based on the obtained autonomic nervous system activity information of the user, and provide input to the sensory stimulator based on the determined sympathetic nervous system information, the provided input causing the sensory stimulator to stop delivering the sensory stimulation to the user based on the determined sympathetic nervous system information.

6. A method for delivering sensory stimulation, the method being implemented by a computer system that comprises one or more physical processors executing computer program instructions that, when executed, perform the method, the method comprising:
obtaining, from a sensor, autonomic nervous system activity information of a user during a sleep session;
determining, using the computer system, parasympathetic nervous system information based on the obtained autonomic nervous system activity information of the user; and
providing, using the computer system, input to a sensory stimulator based on the determined parasympathetic nervous system information, the provided input causing the sensory stimulator to deliver the sensory stimulation to the user based on the determined parasympathetic nervous system information.

7. The method of claim 6, wherein the sensor is selected from the group consisting of a heart rate variability sensor, a respiratory effort sensor, a blood pulse wave sensor, a blood pressure sensor, a body temperature sensor, and a digestive activity sensor.

8. The method of claim 6, wherein the provided input causes the sensory stimulator to deliver the sensory stimulation to the user when the determined parasympathetic nervous system information is above a predetermined threshold, and
further comprising obtaining user input parameter information and determining the predetermined threshold based on the obtained user input parameter information.

9. The method of claim 6, wherein the sensory stimulation is selected from the group consisting of olfactory stimulation, auditory stimulation, visual stimulation, touch stimulation, taste stimulation, haptic stimulation, peripheral stimulation, transcranial stimulation, transcranial magnetic stimulation. electric stimulation and magnetic stimulation.

10. The method of claim 6, further comprising determining sympathetic nervous system information based on the obtained autonomic nervous system activity information of the user, and providing input to the sensory stimulator based on the determined sympathetic nervous system information, the provided input causing the sensory stimulator to stop delivering the sensory stimulation to the user based on the determined sympathetic nervous system information.

11. A system for delivering sensory stimulation, the system comprising:
a means for measuring autonomic nervous system activity information of a user during a sleep session;
a means for delivering sensory stimulation to the user during the sleep session; and
a means for executing machine-readable instructions with at least one physical processor, wherein the machine-readable instructions comprising:
obtaining, from the means for sensing, autonomic nervous system activity information of the user;
determining, using the means for executing, parasympathetic nervous system information based on the obtained autonomic nervous system activity information of the user;
providing, using the means for executing, input to the means for delivering based on the determined parasympathetic nervous system information, the provided input causing the means for delivering to deliver the sensory stimulation to the user based on the determined parasympathetic nervous system information.

12. The system of claim 11, wherein the means for measuring is selected from the group consisting of a heart rate variability sensor, a respiratory effort sensor, a blood pulse wave sensor, a blood pressure sensor, a body temperature sensor, and a digestive activity sensor.

13. The system of claim 11, wherein the provided input causes the means for delivering to deliver the sensory stimulation to the user when the determined parasympathetic nervous system information is above a predetermined threshold, and further comprising obtaining user input parameter information and determining, using the means for executing, the predetermined threshold based on the obtained user input parameter information.

14. The system of claim 11, wherein the sensory stimulation is selected from the group consisting of olfactory stimulation, auditory stimulation, visual stimulation, touch stimulation, taste stimulation, haptic stimulation, peripheral stimulation, transcranial stimulation, transcranial magnetic stimulation. electric stimulation and magnetic stimulation.

15. The system of claim 11, wherein the machine-readable instructions comprising determining sympathetic nervous system information based on the obtained autonomic nervous system activity information of the user, and providing input to the means for delivering based on the determined sympathetic nervous system information, the provided input causing the means for delivering to stop delivering the sensory stimulation to the user based on the determined sympathetic nervous system information.
